(19) United States
(12) Patent Application Publication
(10) Pub. No.: US 2022/0142204 A1
(43) Pub. Date: May 12, 2022

(54) CLAY COMPOSITIONS AND METHODS FOR IMPROVING ANIMAL PERFORMANCE

(71) Applicant: NutriQuest, LLC, Mason City, IA (US)

(72) Inventors: Kim Friesen, Mason City, IA (US); Ran Song, Mason City, IA (US); Chad Hagen, Mason City, IA (US); Jeff Hansen, Mason City, IA (US)

(73) Assignee: NutriQuest, LLC, Mason City, IA (US)

(21) Appl. No.: 17/524,709

(22) Filed: Nov. 11, 2021

Related U.S. Application Data
(60) Provisional application No. 63/112,558, filed on Nov. 11, 2020.

(51) Int. Cl. A23K 20/28; A61K 35/02; A61K 9/00; A23K 50/30

(52) U.S. Cl. CPC A23K 20/28; A23K 50/30; A61K 9/0056; A61K 35/02

(57) ABSTRACT

Feed additive compositions and methods of using the additives to improve the performance of an adult animal are disclosed. Improved performance can include reducing maintenance nutrient requirements, improving growth performance, reducing the energy required for intestinal maintenance, reducing the energy required for weight gain, reduced morbidity, increasing growth rate, improving feed conversion, improving the average daily gain (ADG), improving the efficiency of nutrient utilization, increasing volatile fatty acid production, improving energy use from volatile fatty acids, increasing ending body weight, improved intestinal health, increased relative abundance of Bifidobacterium in the intestines, or combinations thereof.

### CROSS-REFERENCE TO RELATED APPLICATION

This application claims priority to U.S. Provisional Application No. 63/112,558, filed Nov. 11, 2020, the contents of all of which are hereby incorporated by reference in their entirety.

### FIELD OF THE INVENTION

The present disclosure generally relates to feed additive compositions and methods of using the additives to improve the performance in animal production.

### BACKGROUND OF THE INVENTION

The demand for food and food products from animal husbandry is anticipated to increase significantly as the population is growing. Also, with the increase in population, increased demands on land, water, and energy resources are being realized. Global environmental challenges, including global climate changes and the growing threat of disease transmission to and from agricultural animals, add further challenges. Therefore, farmers need to become more efficient, produce the products at a higher rate, and raise livestock and poultry at an increased rate to meet market challenges. Additionally, farmers need a low cost method to produce these products since the profit margin in these areas can be quite low.

To meet these challenges, what is needed is a feed additive which is generally low cost but also promotes health, growth, and reduces the levels of pathogens in animals without having to use antibiotics and hormones.

### SUMMARY OF THE INVENTION

One aspect of the present disclosure encompasses a method of improving performance of an adult animal. Improving performance can comprise reducing maintenance nutrient requirements, improving growth performance, reducing the energy required for intestinal maintenance, reducing the energy required for weight gain, reduced morbidity, increasing growth rate, improving feed conversion, improving the average daily gain (ADG), improving the efficiency of nutrient utilization, increasing volatile fatty acid production, improving energy use from volatile fatty acids, increasing ending body weight, improved intestinal health, increased relative abundance of Bifidobacterium in the intestines, or combinations thereof.

The method comprises orally administrating to the animal a therapeutically effective amount of a therapeutic clay. The therapeutic clay can be clay mined in the Crater Lake region of the Cascade Mountains of Oregon. In some aspects, the therapeutic clay is naturally mined, and the level of reducing agent in the clay is adjusted to provide therapeutically effective amounts of the reducing agent.

The adult animal can be an adult livestock animal. The adult animal can be a grow-finish pig. In some aspects, the adult animal is a healthy grow-finish pig. When the animal is a grow-finish pig, the therapeutic clay can be administered to the pig at or after about week 6 or at about week 8 to about week 17 of growing and finishing. In some aspects, the clay is fed to the pig when the weight of the pig is about 160 to about 200 lbs or about 190 to about 200 lbs.

The clay can be formulated in a feed composition for oral administration to the animal. The amount of clay in the feed composition can range from about 0.005% to about 0.1%, from about 0.01% to about 0.05%, or about 0.01% to about 0.02% of the feed composition. The amount of clay in a feed composition can also range from about 0.01 to about 10 lbs/ton, from about 0.1 to about 1 lbs/ton, or about 0.2 to about 0.6 lbs/ton of the feed composition.

Another aspect of the present disclosure encompasses a feed composition for improving performance of an adult animal. The composition comprises a therapeutically effective amount of a therapeutic clay. The therapeutic clay can be clay mined in the Crater Lake region of the Cascade Mountains of Oregon. In some aspects, the therapeutic clay is naturally mined, and the level of reducing agent in the clay is adjusted to provide antimicrobial effective amounts of the reducing agent.

The adult animal can be an adult livestock animal. The adult animal can be a grow-finish pig. In some aspects, the adult animal is a healthy grow-finish pig. When the animal is a grow-finish pig, the therapeutic clay can be administered to the pig at or after about week 6 or at about week 8 to about week 17 of growing and finishing. In some aspects, the clay is fed to the pig when the weight of the pig is about 160 to about 200 lbs or about 190 to about 200 lbs.

The feed composition can be a basal feed composition, and the amount of clay in a feed composition ranges from about 0.005% to about 0.1%, from about 0.01% to about 0.05%, or about 0.01% to about 0.02% of the feed composition.

### BRIEF DESCRIPTION OF THE FIGURES

FIG. 1 depicts a plot showing the impact of feeding therapeutic clay on enzyme activity of myeloperoxidase in feces.

FIG. 2 depicts a plot showing the impact of feeding therapeutic clay on changes of microflora abundance in finishing pigs.

FIG. 3 depicts a plot showing the impact of feeding therapeutic clay on production of short-chain fatty acids in finishing pigs.

### DETAILED DESCRIPTION OF THE INVENTION

The present disclosure is directed to therapeutic clays and methods of using the therapeutic clays to improve efficiency, growth, and performance in animal production. The inventors discovered that orally administering a therapeutic clay of the instant disclosure to an animal improves the growth performance of the animal. Animals fed diets containing the therapeutic clay of the instant disclosure had reduced maintenance energy requirements, improved intestinal health resulting in greater energy for body weight gain, less dietary energy required for maintenance, and improved feed conversion. Conversely, animals fed diets containing clay other than the therapeutic clay of the instant disclosure did not result in any improved efficiency, growth, or performance of the animal.

Surprisingly and unexpectedly, the low amounts of the therapeutic clay were beneficial even when fed to adult animals. In contrast, adult pigs fed diets containing clay other than the therapeutic clay of the instant disclosure failed to provide any improvements in performance of the animal, even when fed concentrations as high as 15 fold higher than the rate of administration described herein (See, e.g., Subramanian and Kim, Journal of Animal Science and Biotechnology (2015) 6:38, the contents of which are hereby incorporated by reference in their entirety). Any significant effect of feeding a clay other than the therapeutic clay of the instant disclosure was only observed in sub-adult pigs.

More surprisingly, growth performance of the animal was significantly improved even when the effective amounts of the therapeutic clay fed to the animal were many fold lower than effective amounts of clay normally fed to animals before the invention was made. For instance, the inventors discovered that, when fed to pigs, growth performance of the pigs was significantly improved even when administered to animals in feed compositions comprising less than about 0.05 lbs of the therapeutic clay per ton of feed (about 0.05% w/w). In comparison, before the invention was made, at least about 0.5% w/w amounts of clay, a 10 fold higher concentration than the concentration in a feed of the instant disclosure, were normally administered to the sub-adult pig to obtain any benefits (See, e.g., Subramanian and Kim, Journal of Animal Science and Biotechnology (2015) 6:38, the contents of which are hereby incorporated by reference in their entirety). When the weight of adult and sub-adult pigs is taken into consideration, the difference in the rate of administration of clay required to produce significant beneficial effects is even more pronounced, with at least 100 fold higher concentration of clay than the concentration of the therapeutic clay of the instant disclosure.

### I. Therapeutic Clay and Clay Compositions

In one aspect, the present disclosure provides a therapeutic clay and feed or supplement compositions comprising the therapeutic clay. The therapeutic clay may be formulated with nutritive or other pharmaceutical agents for administration to an animal. The therapeutic clay and formulations comprising the therapeutic clay are described below.

### (a) Therapeutic Clay

The term “clay” as used herein refers to a fine-grained natural rock or soil material that combines one or more clay minerals with traces of metal oxides and organic matter. Clays from natural geologic clay deposits are mostly composed of silicate minerals containing variable amounts of water trapped in the mineral structure. Additionally, as it will be recognized by an individual skilled in the art, clays may further comprise various amounts of metal oxides, organic matter, and other materials that can be mixed in with the clay. Clays can comprise varying amounts of iron, magnesium, alkali metals, alkaline earths and other cations. Depending on the content of the soil, clay can appear in various colors, from white to dull gray or brown to a deep orange-red. Clays may be broadly classified into swelling clays, non-swelling clays, and mixed layer clays.

Any clay may be used in a composition or method of the present disclosure, provided the clay has therapeutic properties when administered at the concentrations discovered by the inventors to be beneficial. A clay having therapeutic properties suitable for a method of the instant disclosure can be as described in U.S. patent application Ser. No. 15/266,570, the disclosure of which is incorporated herein by reference in its entirety.

Without wishing to be bound by theory, therapeutic properties of the therapeutic clay may include improved volatile fatty acid (VFA) production, improved intestinal performance resulting in greater energy release from the diet observed in the animals fed the therapeutic clay, improved microflora composition by shifting the microflora to more beneficial bacteria species, increased ability to ferment fiber, and any combination thereof. Therapeutic properties may also include antimicrobial properties, and antitoxin properties. It is noted that, although a therapeutic clay of the instant disclosure can have antimicrobial activity, the beneficial effects described herein do not necessarily result from the antimicrobial activity of the therapeutic clay (see, e.g., Example 4 herein below). Accordingly, in some aspects, a therapeutic clay can have therapeutic properties distinct from antimicrobial and antitoxin activity.

In some aspects, a therapeutic clay can be combined with other clays. For instance, the therapeutic clay can be combined with other clays at a ratio of about 1:99 therapeutic clay to other clays, to about 99:1 therapeutic clay to other clays; at a ratio of about 80:20 therapeutic clay to other clays, to about 30:70 therapeutic clay to other clays; or at a ratio of about 45:55 therapeutic clay to other clays. In some aspects, the therapeutic clay can be combined with a bentonite clay.

A therapeutic clay may be a swelling clay, a non-swelling clay, a mixed layer clay, or a combination of a swelling clay, a non-swelling clay, and a mixed layer clay. In some aspects, the therapeutic clay of the present disclosure is a swelling clay. Swelling or expansive clays are clays prone to large volume changes (swelling and shrinking) that are directly related to changes in water content. Swelling clays are generally referred to as smectite clays. Smectite clays have approximately 1-nm thick 2:1 layers (c-direction of unit cell) separated by hydrated interlayer cations which give rise to the clay's swelling. The “a” and “b” dimensions of the mineral are on the order of several microns. The layers themselves are composed of two opposing silicate sheets, which contain Si and Al in tetrahedral coordination with oxygen, separated by an octahedral sheet that contains Al, Fe and Mg in octahedral coordination with hydroxyls. The surfaces of the 2:1 layers (two tetrahedral sheets with an octahedral sheet in between) carry a net negative charge that is balanced by interlayer cations. The charged surfaces of the 2:1 layers attract cations and water, which leads to swelling.

Smectite clays may be classified with respect to the location of the negative charge on the 2:1 layers, and based on the composition of the octahedral sheet (either dioctahedral or trioctahedral). Dioctahedral smectites include beidellite having the majority of charge in the tetrahedral sheet, and montmorillonite having the majority of charge in the octahedral sheet. Similar trioctahedral smectites are saponite and hectorite. Swelling and other properties of smectite can be altered by exchanging the dominant interlayer cation. For example, swelling can be limited to 2 water layers by exchanging Na for Ca.

Smectite clays may be naturally mined. Alternatively, smectite clays may be synthesized. Methods of synthesizing smectite clays may be as described in U.S. Pat. No. 4,861,584, the disclosure of which is incorporated by reference herein in its entirety.

In other aspects, a therapeutic clay of the present disclosure is a non-swelling clay, also generally known as illite clays. Illite clays are similar in structure to smectite clays, but have their 2:1 layers bound together by poorly hydrated potassium ions, and for that reason do not swell.

In some aspects, a therapeutic clay of the present disclosure is a mixed-layer clay. Mixed-layer clays are generally referred to as rectorite and are composed of ordered mixed layers of illite and smectite. Layers of illite and smectite in rectorite clays may be random or regular. Ordering of illite and smectite layers in rectorite may be referred to as R⁰ ordered or R¹ ordered illite-smectite. R¹-ordered illite-smectite is ordered in an ISISIS fashion, whereas R⁰ describes random ordering. Other advanced ordering types may also be described. In some aspects, a clay of the present disclosure is a rectorite having R¹ ordered layers of illite and smectite.

A therapeutic clay of the present disclosure can be a K-rectorite. In some aspects, the therapeutic clay is a K-rectorite comprising therapeutic effective amounts of a reducing agent. In one aspect, the therapeutic clay is a K-rectorite comprising therapeutic effective amounts of pyrite, or a K-rectorite comprising therapeutic effective amounts of Fe³⁺.

A therapeutic clay of the present disclosure can be an unrefined naturally occurring therapeutic clay. Alternatively, the therapeutic clay may be a refined clay purified from other material normally present in naturally occurring clay. Additionally, a clay may be purified to provide a substantially single form of the therapeutic clay. For instance, when the clay is a rectorite clay, the clay may be purified to provide a substantially pure K-rectorite clay, a substantially pure Na-rectorite clay, or a substantially pure Ca-rectorite clay. In some aspects, the therapeutic clay is a naturally occurring clay. In other aspects, the therapeutic clay is a refined clay. In other aspects, the therapeutic clay is a purified clay.

In some aspects, the therapeutic clay is an unrefined, naturally occurring clay. In another aspect, the therapeutic clay is a refined naturally occurring therapeutic clay. In yet other aspects, the therapeutic clay is synthesized. Methods of synthesizing therapeutic clays may be as described in U.S. Patent Publication No. 2013/0004544, the disclosure of which is incorporated by reference herein in its entirety. In other aspects, therapeutic clays are naturally mined, and the levels of reducing agents in the mined clays are adjusted to provide therapeutic effective amounts of reducing agents in the therapeutic clay.

In some aspects, the therapeutic clay of the present disclosure is a naturally mined clay from an open pit mine in hydrothermally altered, pyroclastic material in the Cascade Mountains. Without wishing to be bound by theory, the therapeutic properties of the therapeutic clay may be due to a rare transition metal combination, including a level of pyrite ranging from about 3% to about 10% wt/wt and/or a level of pyrite ranging from about 1% to about 5% wt/wt.

In other aspects, the therapeutic clay of the present disclosure is a natural red clay mined in the Cascade Mountain region of Oregon, more specifically a red clay mined in the crater lake region of the Cascade Mountains of Oregon. Without wishing to be bound by theory, the therapeutic properties of the red clay may be due to the presence of therapeutic effective amounts of aluminum, among other properties.

The therapeutic clay can also be modified with various substituents to alter the properties of the clay. Non-limiting examples of modifications include modification with organic material, polymers, reducing agents, and various elements such as sodium, iron, silver, or bromide, or by treatment with a strong acid. In some aspects, a therapeutic clay of the present disclosure is modified with reducing metal oxides. In some alternatives of the aspects, when the therapeutic clay is modified with reducing metal oxides, the therapeutic clay is modified with pyrite.

The particle size of the therapeutic clay may be an important factor that can influence its effectiveness, as well as bioavailability, blend uniformity, segregation, and flow properties. In general, smaller particle sizes of clay increase its effectiveness by increasing the surface area. In various aspects, the average particle size of the therapeutic clay is less than about 500 microns in diameter, or less than about 450 microns in diameter, or less than about 400 microns in diameter, or less than about 350 microns in diameter, or less than about 300 microns in diameter, or less than about 250 microns in diameter, or less than about 200 microns in diameter, or less than about 150 microns in diameter, or less than about 100 microns in diameter, or less than about 75 microns in diameter, or less than about 50 microns in diameter, or less than about 25 microns in diameter, or less than about 15 microns in diameter. In some applications, the use of particles less than 15 microns in diameter may be advantageous. The average particle size of the clay can be about 1 to about 200 microns in diameter, or from about 10 to about 150 microns in diameter.

Similarly, in aspects wherein a reducing agent may be added to a therapeutic clay, the particle size of a reducing agent may also be an important factor that can influence its effectiveness, and in general, smaller particle sizes increase its effectiveness. The average particle size of the reducing agent that may be added to the therapeutic clay can be less than 1 micron in size.

### (b) Dietary Supplements or Feed Compositions Comprising a Therapeutic Clay

One aspect of the present disclosure provides dietary supplements or feed compositions comprising a therapeutically effective amount of a therapeutic clay. Formulating the therapeutic clay with other ingredients can facilitate oral administration and effective use of the therapeutic clay.

A therapeutically effective amount of a therapeutic clay in a feed supplement composition can and will vary depending on the therapeutic clay, the body weight, sex, age and/or medical condition of the animal, the method of administration, the duration of treatment, as well as the species of the animal, and can be determined experimentally using methods known in the art.

As described above, it was discovered that the therapeutic clay can be effective at improving growth performance of an animal even when the amounts of the therapeutic clay fed to the animal is many fold lower than amounts of clay normally fed to animals before the disclosure was made. Accordingly, when the therapeutic clay is included in a dietary supplement or feed composition, the therapeutic clay can be included in the composition at less than about 1% w/w, less than about 0.5% w/w, less than about 0.4% w/w, less than about 0.3% w/w, less than about 0.2% w/w, less than about 0.1% w/w, less than about 0.09% w/w, less than about 0.08% w/w, less than about 0.07% w/w, less than about 0.06% w/w, less than about 0.05% w/w, less than about 0.04% w/w, less than about 0.03% w/w, less than about 0.02% w/w, less than about 0.01% w/w, less than about 0.009% w/w, less than about 0.008% w/w, less than about 0.007% w/w, less than about 0.006% w/w, less than about 0.005% w/w, less than about 0.004% w/w, less than about 0.003% w/w, less than about 0.002% w/w, or less than about 0.001% w/w. In some aspects, the therapeutic clay is included in the composition at a concentration ranging from about 0.001% to about 1% w/w, from about 0.005% to about 1% w/w, from about 0.001% to about 0.5% w/w, from about 0.001% to about 0.1% w/w, from about 0.01% to about 0.05% w/w, from about 0.01% to about 0.04% w/w, from about 0.01% to about 0.03% w/w, or at about 0.02% w/w.

When the therapeutic clay is included in a dietary supplement or feed composition, the therapeutic clay can be included in the composition at less than about 10 lbs/ton, less than about 5 lbs/ton, less than about 4 lbs/ton, less than about 3 lbs/ton, less than about 2 lbs/ton, less than about 1 lbs/ton, less than about 0.9 lbs/ton, less than about 0.8 lbs/ton, less than about 0.7 lbs/ton, less than about 0.6 lbs/ton, less than about 0.5 lbs/ton, less than about 0.4 lbs/ton, less than about 0.3 lbs/ton, less than about 0.2 lbs/ton, less than about 0.1 lbs/ton, less than about 0.09 lbs/ton, less than about 0.08 lbs/ton, less than about 0.07 lbs/ton, less than about 0.06 lbs/ton, less than about 0.05 lbs/ton, less than about 0.04 lbs/ton, less than about 0.03 lbs/ton, less than about 0.02 lbs/ton, or less than about 0.01 lbs/ton. In some aspects, the therapeutic clay is included in the composition at a concentration ranging from about 0.01 lbs/ton to about 10 lbs/ton, from about 0.05 lbs/ton to about 10 lbs/ton, from about 0.1 lbs/ton to about 5 lbs/ton, from about 0.1 lbs/ton to about 1 lbs/ton, from about 0.2 lbs/ton to about 1 lbs/ton, from about 0.3 lbs/ton to about 0.5 lbs/ton, or at about 0.4 lbs/ton.

The terms “feed”, “food”, “feed composition”, and “feed supplement”, are used herein interchangeably and may refer to any feed composition normally fed to an animal. Feed compositions normally fed to an animal are known in the art. A feed composition may include one or more components of an animal feed. Non-limiting examples of feed matter or animal feed matter may include, without limitation: corn or a component of corn, such as, for example, corn meal, corn fiber, corn hulls, corn DDGS (distiller's dried grain with solubles), silage, ground corn, corn germ, corn gluten, corn oil, or any other portion of a corn plant; soy or a component of soy, such as, for example, soy oil, soy meal, soy hulls, soy silage, ground soy, or any other portion of a soy plant; wheat or any component of wheat, such as, for example, wheat meal, wheat fiber, wheat hulls, wheat chaff, ground wheat, wheat germ, or any other portion of a wheat plant; canola, such as, for example, canola oil, canola meal, canola protein, canola hulls, ground canola, or any other portion of a canola plant; sunflower or a component of a sunflower plant; sorghum or a component of a sorghum plant; sugar beet or a component of a sugar beet plant; cane sugar or a component of a sugarcane plant; barley or a component of a barley plant; palm oil, palm kernel or a component of a palm plant; glycerol; corn steep liquor; a waste stream from an agricultural processing facility; lecithin; rumen protected fats; molasses; soy molasses; flax; peanuts; peas; oats; grasses, such as orchard grass and fescue; fish meal, meat & bone meal; feather meal; and poultry byproduct meal; and alfalfa and/or clover used for silage or hay, and various combinations of any of the feed ingredients set forth herein, or other feed ingredients generally known in the art. As it will be recognized in the art, a feed composition may further be supplemented with amino acids, vitamins, minerals, and other feed additives such as other types of enzymes, organic acids, essential oils, probiotics, prebiotics, antioxidants, pigments, anti-caking agents, and the like, as described further below.

A feed composition may be formulated for administration to any animal subject. Suitable subjects include all mammals, avian species, and aquaculture. Non-limiting examples of food animals include poultry (e.g., chickens, including broilers, layers, and breeders, ducks, game hens, geese, guinea fowl/hens, quail, and turkeys), beef cattle, dairy cattle, veal, pigs, goats, sheep, bison, and fishes. Suitable companion animals include, but are not limited to, cats, dogs, horses, rabbits, rodents (e.g., mice, rats, hamsters, gerbils, and guinea pigs), hedgehogs, and ferrets. Examples of research animals include rodents, cats, dogs, rabbits, pigs, and non-human primates. Non-limiting examples of suitable zoo animals include non-human primates, lions, tigers, bears, elephants, giraffes, and the like. In some aspects, the animal is a pig. In other aspects, the animal is an adult pig. In additional aspects, the animal is a growing and finishing pig. As used herein, the term “growing and finishing pig” refers to a weened pig removed from the nursery, at about 6 weeks after birth. In some aspects, the animal is a pig, wherein the weight of the pig is about 110 lbs or more, about 110 to about 400 lbs, about 120 to about 350 lbs, about 150 to about 310 lbs, or about 160 to about 200 lbs.

In some aspects, a feed composition of the instant disclosure is formulated for adult animals. In other aspects, the feed composition is formulated for growing and finishing animals. In yet other aspects, the feed composition is a basal feed composition. In additional aspects, the feed composition is formulated for pigs. In some aspects, the feed composition is formulated for adult pigs. In some aspects, the feed composition is formulated for growing and finishing pigs. In yet other aspects, the feed composition is a basal feed composition formulated for adult pigs. In some aspects, the feed composition is a basal feed composition formulated for growing and finishing pigs.

According to various aspects of the present disclosure, the feed may be in any suitable form known in the animal feed art and may be a wet or dry component. For example, according to certain aspects, the feed composition may be in a form selected from the group consisting of a complete feed, a feed supplement, a feed additive, a premix, a top-dress, a tub, a mineral, a meal, a block, a pellet, a mash, a liquid supplement, a drench, a bolus, a treat, and combinations of any thereof. Additionally, a feed sample may optionally be ground before preparing a feed composition.

The dietary supplements or feed compositions may optionally comprise at least one additional nutritive and/or pharmaceutical agent. For instance, the at least one additional nutritive and/or pharmaceutical agent may be selected from the group consisting of vitamin, mineral, amino acid, antioxidant, probiotic, essential fatty acid, and pharmaceutically acceptable excipient. The compositions may include one additional nutritive and/or pharmaceutical component or a combination of any of the foregoing additional components in varying amounts. Suitable examples of each additional component are detailed below.

A. Vitamins

Optionally, the dietary supplement of the disclosure may include one or more vitamins. Suitable vitamins for use in the dietary supplement include vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

The dietary supplement may include one or more forms of an effective amount of any of the vitamins described herein or otherwise known in the art. Non-limiting examples of vitamins include vitamin K, vitamin D, vitamin C, and biotin. An “effective amount” of a vitamin typically quantifies an amount at least about 10% of the United States Recommended Daily Allowance (“RDA”) of that particular vitamin for a subject. It is contemplated, however, that amounts of certain vitamins exceeding the RDA may be beneficial for certain subjects. For example, the amount of a given vitamin may exceed the applicable RDA by 100%, 200%, 300%, 400%, 500% or more.

B. Minerals

In addition to the metal chelates or metal salts described in Section IA, the dietary supplement may include one or more minerals or mineral sources. Non-limiting examples of minerals include, without limitation, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In an aspect, the mineral may be a form of calcium. Suitable forms of calcium include calcium alpha-ketoglutarate, calcium acetate, calcium alginate, calcium ascorbate, calcium aspartate, calcium caprylate, calcium carbonate, calcium chelates, calcium chloride, calcium citrate, calcium citrate malate, calcium formate, calcium glubionate, calcium glucoheptonate, calcium gluconate, calcium glutarate, calcium glycerophosphate, calcium lactate, calcium lysinate, calcium malate, calcium orotate, calcium oxalate, calcium oxide, calcium pantothenate, calcium phosphate, calcium pyrophosphate, calcium succinate, calcium sulfate, calcium undecylenate, coral calcium, dicalcium citrate, dicalcium malate, dihydroxycalcium malate, dicalcium phosphate, and tricalcium phosphate.

Generally speaking, the dietary supplement may include one or more forms of an effective amount of any of the minerals described herein or otherwise known in the art. An “effective amount” of a mineral typically quantifies an amount at least about 10% of the United States Recommended Daily Allowance (“RDA”) of that particular mineral for a subject. It is contemplated, however, that amounts of certain minerals exceeding the RDA may be beneficial for certain subjects. For example, the amount of a given mineral may exceed the applicable RDA by 100%, 200%, 300%, 400%, 500% or more. Typically, the amount of mineral included in the dietary supplement may range from about 1 mg to about 1500 mg, about 5 mg to about 500 mg, or from about 50 mg to about 500 mg per dosage.

C. Essential fatty acids

Optionally, the dietary supplement may include a source of an essential fatty acid. The essential fatty acid may be isolated or it may be an oil source or fat source that contains an essential fatty acid. In one aspect, the essential fatty acid may be a polyunsaturated fatty acid (PUFA), which has at least two carbon-carbon double bonds generally in the cis-configuration. The PUFA may be a long chain fatty acid having at least 18 carbons atoms. The PUFA may be an omega-3 fatty acid in which the first double bond occurs in the third carbon-carbon bond from the methyl end of the carbon chain (i.e., opposite the carboxyl acid group). Examples of omega-3 fatty acids include alpha-linolenic acid (18:3, ALA), stearidonic acid (18:4), eicosatetraenoic acid (20:4), eicosapentaenoic acid (20:5; EPA), docosatetraenoic acid (22:4), n-3 docosapentaenoic acid (22:5; n-3DPA), and docosahexaenoic acid (22:6; DHA). The PUFA may also be an omega-5 fatty acid, in which the first double bond occurs in the fifth carbon-carbon bond from the methyl end. Non-limiting examples of omega-5 fatty acids include myristoleic acid (14:1), myristoleic acid esters, and cetyl myristoleate. The PUFA may also be an omega-6 fatty acid, in which the first double bond occurs in the sixth carbon-carbon bond from the methyl end. Examples of omega-6 fatty acids include linoleic acid (18:2), gamma-linolenic acid (18:3), eicosadienoic acid (20:2), dihomo-gamma-linolenic acid (20:3), arachidonic acid (20:4), docosadienoic acid (22:2), adrenic acid (22:4), and n-6 docosapentaenoic acid (22:5). The fatty acid may also be an omega-9 fatty acid, such as oleic acid (18:1), eicosenoic acid (20:1), mead acid (20:3), erucic acid (22:1), and nervonic acid (24:1).

In another aspect, the essential fatty acid source may be a seafood-derived oil. The seafood may be a vertebrate fish or a marine organism, such that the oil may be fish oil or marine oil. The long chain (20C, 22C) omega-3 and omega-6 fatty acids are found in seafood. The ratio of omega-3 to omega-6 fatty acids in seafood ranges from about 8:1 to 20:1. Seafood from which oil rich in omega-3 fatty acids may be derived includes, but is not limited to, abalone scallops, albacore tuna, anchovies, catfish, clams, cod, gem fish, herring, lake trout, mackerel, menhaden, orange roughy, salmon, sardines, sea mullet, sea perch, shark, shrimp, squid, trout, and tuna.

In yet another aspect, the essential fatty acid source may be a plant-derived oil. Plant and vegetable oils are rich in omega-6 fatty acids. Some plant-derived oils, such as flaxseed oil, are especially rich in omega-3 fatty acids. Plant or vegetable oils are generally extracted from the seeds of a plant, but may also be extracted from other parts of the plant. Plant or vegetable oils that are commonly used for cooking or flavoring include, but are not limited to, acai oil, almond oil, amaranth oil, apricot seed oil, argan oil, avocado seed oil, babassu oil, ben oil, blackcurrant seed oil, Borneo tallow nut oil, borage seed oil, buffalo gourd oil, canola oil, carob pod oil, cashew oil, castor oil, coconut oil, coriander seed oil, corn oil, cottonseed oil, evening primrose oil, false flax oil, flax seed oil, grapeseed oil, hazelnut oil, hemp seed oil, kapok seed oil, lallemantia oil, linseed oil, macadamia oil, meadowfoam seed oil, mustard seed oil, okra seed oil, olive oil, palm oil, palm kernel oil, peanut oil, pecan oil, pequi oil, perilla seed oil, pine nut oil, pistachio oil, poppy seed oil, prune kernel oil, pumpkin seed oil, quinoa oil, ramtil oil, rice bran oil, safflower oil, sesame oil, soybean oil, sunflower oil, tea oil, thistle oil, walnut oil, or wheat germ oil. The plant-derived oil may also be hydrogenated or partially hydrogenated.

In still a further aspect, the essential fatty acid source may be an algae-derived oil. Commercially available algae-derived oils include those from Crypthecodinium cohnii and Schizochytrium sp. Other suitable species of algae, from which oil is extracted, include Aphanizomenon flos-aquae, Bacilliarophy sp., Botryococcus braunii, Chlorophyceae sp., Dunaliella tertiolecta, Euglena gracilis, Isochrysis galbana, Nannochloropsis saline, Nannochloris sp., Neochloris oleoabundans, Phaeodactylum tricornutum, Pleurochrysis carterae, Prymnesium parvum, Scenedesmus dimorphus, Spirulina sp., and Tetraselmis chui.

D. Amino acids

The dietary supplement may optionally include from one to several amino acids. Suitable amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine or their hydroxy analogs. In certain aspects, the amino acid will be selected from the essential amino acids. An essential amino acid is generally described as one that cannot be synthesized de novo by the organism, and therefore, must be provided in the diet. By way of non-limiting example, the essential amino acids for humans include: L-histidine, L-isoleucine, L-leucine, L-lysine, L-methionine, L-phenylalanine, L-valine and L-threonine.

E. Antioxidants

The dietary supplement may include one or more suitable antioxidants. As will be appreciated by a skilled artisan, the suitability of a given antioxidant will vary depending upon the species to which the dietary supplement will be administered. Non-limiting examples of antioxidants include ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, p-coumaric acid, curcurin, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, propyl gallate, polyphosphates, quercetin, trans-resveratrol, rosmarinic acid, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, zeaxanthin, or combinations thereof.

Natural antioxidants that may be included in the dietary supplement include, but are not limited to, apple peel extract, blueberry extract, carrot juice powder, clove extract, coffeeberry, coffee bean extract, cranberry extract, eucalyptus extract, ginger powder, grape seed extract, green tea, olive leaf, parsley extract, peppermint, pimento extract, pomace, pomegranate extract, rice bran extract, rosehips, rosemary extract, sage extract, tart cherry extract, tomato extract, turmeric, and wheat germ oil.

F. Anti-inflammatory agents

The dietary supplement may optionally include at least one anti-inflammatory agent. In one aspect, the anti-inflammatory agent may be a synthetic non-steroidal anti-inflammatory drug (NSAID) such as acetylsalicylic acid, dichlophenac, indomethacin, oxamethacin, ibuprofen, indoprofen, naproxen, ketoprofen, mefamanic acid, metamizole, piroxicam, and celecoxib. In an alternate aspect, the anti-inflammatory agent may be a prohormone that modulates inflammatory processes. Suitable prohormones having this property include prohormone convertase 1, proopiomelanocortin, prohormone B-type natriuretic peptide, SMR1 prohormone, and the like. In another aspect, the anti-inflammatory agent may be an enzyme having anti-inflammatory effects. Examples of anti-inflammatory enzymes include bromelain, papain, serrapeptidase, and proteolytic enzymes such as pancreatin (a mixture of trypsin, amylase and lipase).

In still another aspect, the anti-inflammatory agent may be a peptide with anti-inflammatory effects. For example, the peptide may be an inhibitor of phospholipase A2, such as antiflammin-1, a peptide that corresponds to amino acid residues 246-254 of lipocortin; antiflammin-2, a peptide that corresponds to amino acid residues 39-47 of uteroglobin; S7 peptide, which inhibits the interaction between interleukin 6 and interleukin 6 receptor; RP1, a prenyl protein inhibitor; and similar peptides. Alternatively, the anti-inflammatory peptide may be cortistatin, a cyclic neuropeptide related to somatostatin, or peptides that correspond to an N-terminal fragment of SV-IV protein, a conserved region of E-, L-, and P-selectins, and the like. Other suitable anti-inflammatory preparations include collagen hydrolysates and milk micronutrient concentrates (e.g., MicroLactin® available from Stolle Milk Biologics, Inc., Cincinnati, Ohio), as well as milk protein hydrolysates, casein hydrolysates, whey protein hydrolysates, and plant protein hydrolysates.

In a further aspect, the anti-inflammatory agent may be a probiotic that has been shown to modulate inflammation. Suitable immunomodulatory probiotics include lactic acid bacteria such as acidophilli, lactobacilli, and bifidophilli. In yet another aspect, the anti-inflammatory agent may be a plant extract having anti-inflammatory properties. Non-limiting examples of suitable plant extracts with anti-inflammatory benefits include blueberries, boswella, black catechu and Chinese skullcap, celery seed, chamomile, cherries, devils claw, eucalyptus, evening primrose, ginger, hawthorne berries, horsetail, Kalopanax pictus bark, licorice root, turmeric, white wallow, willow bark, and yucca.

G. Probiotics

Probiotics and prebiotics may include yeast and bacteria that help establish an immune protective rumen or gut microflora as well as small oligosaccharides. By way of non-limiting example, yeast-derived probiotics and prebiotics include yeast cell wall derived components such as β-glucans, arabinoxylan isomaltose, agarooligosaccharides, lactosucrose, cyclodextrins, lactose, fructooligosaccharides, laminariheptaose, lactulose, β-galactooligosaccharides, mannanoligosaccharides, raffinose, stachyose, oligofructose, glucosyl sucrose, sucrose thermal oligosaccharide, isomalturose, caramel, inulin, and xylooligosaccharides. In one aspect, the yeast-derived agent may be β-glucans and/or mannanoligosaccharides. Sources for yeast cell wall derived components include Saccharomyces bisporus, Saccharomyces boulardii, Saccharomyces cerevisiae, Saccharomyces capsularis, Saccharomyces delbrueckii, Saccharomyces fermentati, Saccharomyces lugwigii, Saccharomyces microellipsoides, Saccharomyces pastorianus, Saccharomyces rosei, Candida albicans, Candida cloaceae, Candida tropicalis, Candida utilis, Geotrichum candidum, Hansenula americana, Hansenula anomala, Hansenula wingei, and Aspergillus oryzae.

Probiotics and prebiotics may also include bacteria cell wall derived agents such as peptidoglycan and other components derived from gram-positive bacteria with a high content of peptidoglycan. Non-limiting examples of gram-positive bacteria include Lactobacillus acidophilus, Bifedobact thermophilum, Bifedobat longhum, Streptococcus faecium, Bacillus pumilus, Bacillus subtilis, Bacillus licheniformis, Lactobacillus acidophilus, Lactobacillus casei, Enterococcus faecium, Bifidobacterium bifidium, Propionibacterium acidipropionici, Propionibacteriium freudenreichii, and Bifidobacterium pseudolongum.

H. Herbals

Suitable herbals and herbal derivatives, as used herein, refer to herbal extracts, and substances derived from plants and plant parts, such as leaves, flowers and roots, without limitation. Non-limiting exemplary herbals and herbal derivatives include agrimony, alfalfa, aloe vera, amaranth, angelica, anise, barberry, basil, bayberry, bee pollen, birch, bistort, blackberry, black cohosh, black walnut, blessed thistle, blue cohosh, blue vervain, boneset, borage, buchu, buckthorn, bugleweed, burdock, phytogenic, cayenne, caraway, cascara sagrada, catnip, celery, centaury, chamomile, chaparral, chickweed, chicory, chinchona, cloves, coltsfoot, comfrey, cornsilk, couch grass, cramp bark, culver's root, cyani, cornflower, damiana, dandelion, devils claw, dong quai, echinacea, elecampane, ephedra, eucalyptus, evening primrose, eyebright, false unicorn, fennel, fenugreek, figwort, flaxseed, garlic, gentian, ginger, ginseng, golden seal, gotu kola, gum weed, hawthorn, hops, horehound, horseradish, horsetail, hoshouwu, hydrangea, hyssop, iceland moss, irish moss, jojoba, juniper, kelp, lady's slipper, lemon grass, licorice, lobelia, mandrake, marigold, marjoram, marshmallow, mistletoe, mullein, mustard, myrrh, nettle, oatstraw, oregon grape, papaya, parsley, passion flower, peach, pennyroyal, peppermint, periwinkle, plantain, pleurisy root, pokeweed, prickly ash, psyllium, quassia, queen of the meadow, red clover, red raspberry, redmond clay, rhubarb, rose hips, rosemary, rue, safflower, saffron, sage, St. John's wort, sarsaparilla, sassafras, saw palmetto, skullcap, senega, senna, shepherd's purse, slippery elm, spearmint, spikenard, squawvine, stillingia, strawberry, taheebo, thyme, uva ursi, valerian, violet, watercress, white oak bark, white pine bark, wild cherry, wild lettuce, wild yam, willow, wintergreen, witch hazel, wood betony, wormwood, yarrow, yellow dock, yerba santa, yucca and combinations thereof.

I. Pigments

Suitable non-limiting pigments include actinioerythrin, alizarin, alloxanthin, β-apo-2′-carotenal, apo-2-lycopenal, apo-6′-lycopenal, astacein, astaxanthin, azafrinaldehyde, aacterioruberin, aixin, α-carotine, β-carotine, γ-carotine, β-carotenone, canthaxanthin, capsanthin, capsorubin, citranaxanthin, citroxanthin, crocetin, crocetinsemialdehyde, crocin, crustaxanthin, cryptocapsin, α-cryptoxanthin, β-cryptoxanthin, cryptomonaxanthin, cynthiaxanthin, decaprenoxanthin, dehydroadonirubin, diadinoxanthin, 1,4-diamino-2,3-dihydroanthraquinone, 1,4-dihydroxyanthraquinone, 2,2′-Diketospirilloxanthin, eschscholtzxanthin, eschscholtzxanthone, flexixanthin, foliachrome, fucoxanthin, gazaniaxanthin, hexahydrolycopene, hopkinsiaxanthin, hydroxyspheriodenone, isofucoxanthin, loroxanthin, lutein, luteoxanthin, lycopene, lycopersene, lycoxanthin, morindone, mutatoxanthin, neochrome, neoxanthin, nonaprenoxanthin, OH-Chlorobactene, okenone, oscillaxanthin, paracentrone, pectenolone, pectenoxanthin, peridinin, phleixanthophyll, phoeniconone, phoenicopterone, phoenicoxanthin, physalien, phytofluene, pyrrhoxanthininol, quinones, rhodopin, rhodopinal, rhodopinol, rhodovibrin, rhodoxanthin, rubixanthone, saproxanthin, semi-α-carotenone, semi-β-carotenone, sintaxanthin, siphonaxanthin, siphonein, spheroidene, tangeraxanthin, torularhodin, torularhodin methyl ester, torularhodinaldehyde, torulene, 1,2,4-trihydroxyanthraquinone, triphasiaxanthin, trollichrome, vaucheriaxanthin, violaxanthin, wamingone, xanthin, zeaxanthin, α-zeacarotene and combinations thereof.

J. Pharmaceutical Agents

Suitable non-limiting pharmaceutically acceptable agents include an acid/alkaline-labile drug, a pH dependent drug, or a drug that is a weak acid or a weak base. Examples of acid-labile drugs include statins (e.g., pravastatin, fluvastatin and atorvastatin), antibiotics (e.g., penicillin G, ampicillin, streptomycin, erythromycin, clarithromycin and azithromycin), nucleoside analogs (e.g., dideoxyinosine (ddI or didanosine), dideoxyadenosine (ddA), dideoxycytosine (ddC)), salicylates (e.g., aspirin), digoxin, bupropion, pancreatin, midazolam, and methadone. Drugs that are only soluble at acid pH include nifedipine, emonapride, nicardipine, amosulalol, noscapine, propafenone, quinine, dipyridamole, josamycin, dilevalol, labetalol, enisoprost, and metronidazole. Drugs that are weak acids include phenobarbital, phenytoin, zidovudine (AZT), salicylates (e.g., aspirin), propionic acid compounds (e.g., ibuprofen), indole derivatives (e.g., indomethacin), fenamate compounds (e.g., meclofenamic acid), pyrrolealkanoic acid compounds (e.g., tolmetin), cephalosporins (e.g., cephalothin, cephalaxin, cefazolin, cephradine, cephapirin, cefamandole, and cefoxitin), 6-fluoroquinolones, and prostaglandins. Drugs that are weak bases include adrenergic agents (e.g., ephedrine, desoxyephedrine, phenylephrine, epinephrine, salbutamol, and terbutaline), cholinergic agents (e.g., physostigmine and neostigmine), antispasmodic agents (e.g., atropine, methantheline, and papaverine), curariform agents (e.g., chlorisondamine), tranquilizers and muscle relaxants (e.g., fluphenazine, thioridazine, trifluoperazine, chlorpromazine, and triflupromazine), antidepressants (e.g., amitriptyline and nortriptyline), antihistamines (e.g., diphenhydramine, chlorpheniramine, dimenhydrinate, tripelennamine, perphenazine, chlorprophenazine, and chlorprophenpyridamine), cardioactive agents (e.g., verapamil, diltiazem, gallapomil, cinnarizine, propranolol, metoprolol and nadolol), antimalarials (e.g., chloroquine), analgesics (e.g., propoxyphene and meperidine), antifungal agents (e.g., ketoconazole and itraconazole), antimicrobial agents (e.g., cefpodoxime, proxetil, and enoxacin), caffeine, theophylline, and morphine. In another aspect, the drug may be a biphosphonate or another drug used to treat osteoporosis. Non-limiting examples of a biphosphonate include alendronate, ibandronate, risedronate, zoledronate, pamidronate, neridronate, olpadronate, etidronate, clodronate, and tiludronate. Other suitable drugs include estrogen, selective estrogen receptor modulators (SERMs), and parathyroid hormone (PTH) drugs. In yet another aspect, the drug may be an antibacterial agent (antibiotic). Suitable antibiotics include aminoglycosides (e.g., amikacin, gentamicin, kanamycin, neomycin, netilmicin, streptomycin, and tobramycin), carbecephems (e.g., loracarbef), a carbapenem (e.g., certapenem, imipenem, and meropenem), cephalosporins (e.g., cefadroxil cefazolin, cephalexin, cefaclor, cefamandole, cephalexin, cefoxitin, cefprozil, cefuroxime, cefixime, cefdinir, cefditoren, cefoperazone, cefotaxime, cefpodoxime, ceftazidime, ceftibuten, ceftizoxime, and ceftriaxone), macrolides (e.g., azithromycin, clarithromycin, dirithromycin, erythromycin, and troleandomycin), monobactam, penicillins (e.g., amoxicillin, ampicillin, carbenicillin, cloxacillin, dicloxacillin, nafcillin, oxacillin, penicillin G, penicillin V, piperacillin, and ticarcillin), polypeptides (e.g., bacitracin, colistin, and polymyxin B), quinolones (e.g., ciprofloxacin, enoxacin, gatifloxacin, levofloxacin, lomefloxacin, moxifloxacin, norfloxacin, ofloxacin, and trovafloxacin), sulfonamides (e.g., mafenide, sulfacetamide, sulfamethizole, sulfasalazine, sulfisoxazole, and trimethoprim-sulfamethoxazole), and tetracyclines (e.g., demeclocycline, doxycycline, minocycline, and oxytetracycline). In an alternate aspect, the drug may be an antiviral protease inhibitor (e.g., amprenavir, fosamprenavir, indinavir, lopinavir/ritonavir, ritonavir, saquinavir, and nelfinavir). In still another aspect, the drug may be a cardiovascular drug. Examples of suitable cardiovascular agents include cardiotonic agents (e.g., digitalis (digoxin), ubidecarenone, and dopamine), vasodilating agents (e.g., nitroglycerin, captopril, dihydralazine, diltiazem, and isosorbide dinitrate), antihypertensive agents (e.g., alpha-methyldopa, chlortalidone, reserpine, syrosingopine, rescinnamine, prazosin, phentolamine, felodipine, propanolol, pindolol, labetalol, clonidine, captopril, enalapril, and lisonopril), beta blockers (e.g., levobunolol, pindolol, timolol maleate, bisoprolol, carvedilol, and butoxamine), alpha blockers (e.g., doxazosin, prazosin, phenoxybenzamine, phentolamine, tamsulosin, alfuzosin, and terazosin), calcium channel blockers (e.g., amlodipine, felodipine, nicardipine, nifedipine, nimodipine, nisoldipine, nitrendipine, lacidipine, lercanidipine, verapamil, gallopamil, and diltiazem), and anticlot agents (e.g., dipyrimadole).

K. Excipients

A variety of commonly used excipients in dietary supplement formulations may be selected on the basis of compatibility with the active ingredients. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste-masking agents, an effervescent disintegration agent, and combinations of any of these agents.

In one aspect, the excipient is a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons.

In another aspect, the excipient may be a filler. Suitable fillers include carbohydrates, inorganic compounds, and polyvinylpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In another aspect, the excipient may be an effervescent disintegrant. By way of non-limiting example, suitable effervescent disintegrants include sodium bicarbonate in combination with citric acid and sodium bicarbonate in combination with tartaric acid.

The excipient may comprise a preservative. Suitable examples of preservatives include antioxidants, such as a-tocopherol or ascorbate, and antimicrobials, such as parabens, chlorobutanol or phenol.

In another aspect, the excipient may include a diluent. Diluents suitable for use include pharmaceutically acceptable saccharide such as sucrose, dextrose, lactose, microcrystalline cellulose, fructose, xylitol, and sorbitol; polyhydric alcohols; a starch; pre-manufactured direct compression diluents; and mixtures of any of the foregoing.

The excipient may include flavors. Flavors incorporated into the outer layer may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot.

In another aspect, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevie Rebaudiana (Stevioside); chloro derivatives of sucrose such as sucralose; sugar alcohols such as sorbitol, mannitol, sylitol, and the like.

In another aspect, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Depending upon the aspect, it may be desirable to provide a coloring agent in the outer layer. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in the present disclosure depending on the aspect.

The excipient may include a taste-masking agent. Taste-masking materials include, e.g., cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking materials contemplated are those described in U.S. Pat. Nos. 4,851,226, 5,075,114, and 5,876,759, each of which is hereby incorporated by reference in its entirety.

In various aspects, the excipient may include a pH modifier. In certain aspects, the pH modifier may include sodium carbonate or sodium bicarbonate.

The dietary supplement or feed compositions detailed herein may be manufactured in one or several dosage forms. In one aspect, the dosage form will be an oral dosage form. Suitable oral dosage forms may include a tablet, for example a suspension tablet, a chewable tablet, an effervescent tablet or caplet; a pill; a powder, such as a sterile packaged powder, a dispensable powder, and an effervescent powder; a capsule including both soft or hard gelatin capsules or non-animal derived polymers, such as hydroxypropyl methylcellulose capsules (i.e., HPMC) or pullulan; a lozenge; a sachet; a sprinkle; a reconstitutable powder or shake; a troche; pellets; granules; liquids; lick blocks; suspensions; emulsions; or semisolids and gels. Alternatively, the dietary supplement may be incorporated into a food product or powder for mixing with a liquid, or administered orally after only mixing with a non-foodstuff liquid. As will be appreciated by a skilled artisan, the dietary supplements, in addition to being suitable for administration in multiple dosage forms, may also be administered with various dosage regimens. Additionally, the therapeutic clay may simply be added to any dosage form of a dietary supplement or feed composition.

The amount and types of ingredients (i.e., metal chelate, chondroprotective agents, vitamin, mineral, amino acid, antioxidant, yeast culture, and essential fatty acid), and other excipients useful in each of these dosage forms, are described throughout the specification and examples. It should be recognized that where a combination of ingredients and/or excipient, including specific amounts of these components, is described with one dosage form that the same combination could be used for any other suitable dosage form. Moreover, it should be understood that one of skill in the art would, with the teachings found within this application, be able to make any of the dosage forms listed above by combining the amounts and types of ingredients administered as a combination in a single dosage form or separate dosage forms and administered together as described in the different sections of the specification.

The dietary supplements of the present disclosure can be manufactured by conventional pharmacological techniques. Conventional pharmacological techniques include, e.g., one or a combination of methods: (1) dry mixing; (2) direct compression; (3) milling; (4) dry or non-aqueous granulation; (5) wet granulation; or (6) fusion. See, e.g., Lachman et al., The Theory and Practice of Industrial Pharmacy (1986). Other methods include, e.g., prilling, spray drying, pan coating, melt granulation, granulation, wurster coating, tangential coating, top spraying, extruding, coacervation and the like.

### II. Methods of Using

Another aspect of the present disclosure provides methods of improving the performance of an animal by orally administering to the animal an effective amount of a therapeutic clay. The therapeutic clay can be as described in Section I(a) herein above. In some aspects, the method comprises orally administering to the animal an effective amount of a therapeutic clay formulated in a dietary supplement or feed composition. Dietary supplements or feed compositions comprising a therapeutic clay can be as described in Section I (b) herein above.

As used herein, the term “improving performance” refers to any significant improvement in the performance of an animal obtained when the animal is administered therapeutically effective amounts of the therapeutic clay. The improvement in performance can comprise a reduction in maintenance nutrient requirements, an improvement in growth performance, a reduction in the energy required for intestinal maintenance, a reduction in the energy required for weight gain, a reduction in morbidity, an increase in growth rate, an improvement in feed conversion, improving the average daily gain (ADG), improving the efficiency of nutrient utilization, an improvement in volatile fatty acid production, an improvement in energy use from volatile fatty acids, an increase in ending body weight, an improvement in intestinal health, an improvement in gut microflora composition, an increase in relative abundance of Bifidobacterium in the intestines, or combinations thereof.

A therapeutically effective amount of the therapeutic clay is any amount of the therapeutic clay that, when administered to an animal, will improve the performance of the animal when compared to the performance of an animal fed a control diet without the therapeutic clay. The therapeutic clay or any combination of the therapeutic clay with other ingredients can be used for oral administration.

Any method of oral administration can be used, provided the method is a controlled method of administration capable of administering an accurate amount of therapeutic clay to the animal. For instance, the therapeutic clay can be administered by sprinkling an accurate amount of therapeutic clay over a feed composition (topping off) or by adding to drinking water to administer the accurate amount of therapeutic clay upon ingestion of the feed or water by the animal. Alternatively, the therapeutic clay can be formulated with a feed composition to administer the accurate amount of therapeutic clay upon ingestion of the feed composition by the animal.

An animal can include, without limitation, companion animals such as cats, dogs, rabbits, horses, and rodents such as gerbils; agricultural animals such as cows, dairy cows, dairy calves, beef cattle, pigs, goats, sheep, horses, deer; zoo animals such as primates, elephants, zebras, large cats, bears, and the like; research animals such as rabbits, sheep, pigs, dogs, primates, mice, rats and other rodents; avians, including but not limited to chickens, ducks, turkeys, ostrich, and emu; and aquatic animals chosen from fish and crustaceans including, but not limited to, salmon, shrimp, carp, tilapia, and shell fish.

In some aspects, the animal is a livestock animal. The term “livestock” as used herein refers to domesticated animals raised in an agricultural setting to produce labor and commodities such as meat, eggs, milk, fur, leather, and wool. The term “livestock” can be used to refer solely to animals that are bred for consumption. The term can also be used to refer only to farmed mammalian animals, such as cattle, sheep, horses, pigs, and goats. Other animals may be as described in Section I (b) herein above.

In some aspects, the animal is a pig. In some aspects, the animal is an adult pig. In some aspects, the animal is a growing and finishing pig. In some aspects, the animal is a pig, wherein the weight of the pig is about 110 lbs or more, about 110 to about 400 lbs, about 120 to about 350 lbs, about 150 to about 310 lbs, or about 160 to about 200 lbs.

The timing and duration of administration of the therapeutic clay of the disclosure to an animal can and will vary. For instance, a therapeutic clay can be administered routinely throughout the period when the animal is raised. In some aspects, the therapeutic clay is administered to growing and finishing animals. A therapeutic clay can be administered at various intervals. For instance, a therapeutic clay can be administered daily, weekly, monthly or over a number of months. In some aspects, a therapeutic clay is administered daily. In other aspects, a therapeutic clay is administered weekly. In yet other aspects, a therapeutic clay is administered monthly. A therapeutic clay can also be administered every three to six months. As it will be recognized in the art, the duration of treatment can and will vary and can be determined experimentally.

The timing and duration of administration of the therapeutic clay can be determined based on the growing stage of the animal. In some aspects, the therapeutic clay is administered during growing and finishing of the animal. For instance, when the animal is a pig, a therapeutic clay can be administered at or after about week 6, at or after about week 7, at or after about week 8, at or after about week 6 to about week 20, at about week 7 to about week 19, or at about week 8 to about week 17 of the growing and finishing period. In some aspects, the therapeutic clay is administered throughout the growing and farrowing period of a pig. In some aspects, the therapeutic clay is administered at about week 8 to about week 17 of the growing and finishing period of the pig.

Alternatively, the timing and duration of administration of the therapeutic clay can be determined based on the weight of the animal. For instance, when the animal is a pig, a therapeutic clay can be administered when the weight of the animal is about 110 lbs or more, about 110 to about 400 lbs, about 120 to about 350 lbs, about 150 to about 310 lbs, or about 160 to about 200 lbs. In some aspects, therapeutic clay can be administered when the weight of the animal is about 110 lbs or more. In some aspects, therapeutic clay can be administered when the weight of the animal is about about 110 to about 400 lbs. In some aspects, therapeutic clay can be administered when the weight of the animal is about 120 to about 350 lbs. In some aspects, therapeutic clay can be administered when the weight of the animal is about 150 to about 310 lbs. In some aspects, therapeutic clay can be administered when the weight of the animal is about 160 to about 200 lbs. In some aspects, therapeutic clay can be administered when the weight of the animal is about 190 to about 200 lbs.

The therapeutic clay can be administered to the animal in a single dose or a number of doses throughout the period of administration. For instance, a single dose or a number of doses of the therapeutic clay can be administered after breeding, a single dose or a number of doses can be administered during gestation, a single dose or a number of doses can be administered at birth, a single dose or a number of doses can be administered after farrowing, a single dose or a number of doses can be administered during growing and finishing, or any combination thereof.

In some aspects, the therapeutic clay is administered orally to an animal by adding the therapeutic clay to a feed, formulating the therapeutic clay with the feed, or supplement formulation and feeding the feed or supplement formulation to the animal. Formulating the therapeutic clay with a feed can be as described in Section I (b) above.

As will be appreciated by one of skill in the art, a dose of a composition of the disclosure can and will vary depending on the animal, the frequency and timing of administration of the dose, body weight, sex, age and/or medical condition of the animal, the desired growth rate and efficiency, the method of administration, and the duration of treatment.

The rate of administration of the therapeutic clay of the disclosure may depend on the level of reducing agent in the therapeutic clay. For instance, the level of reducing agent in the therapeutic clay may be determined before administration to adjust the level of therapeutic clay that may be used. The oxidation-reduction potential of the therapeutic clay can be determined and the level of therapeutic clay used in a method, composition, or formulation of the present disclosure is adjusted based on the oxidation-reduction potential of the therapeutic clay. The oxidation-reduction potential of the therapeutic clay can provide a general measure of the therapeutic potential of a therapeutic clay that may be used irrespective of the reducing agents present in the therapeutic clay. Alternatively, the content of one or more specific reducing agents in the therapeutic clay may be determined.

When the animal is a growing and finishing pig, the therapeutic clay can be administered to the pig at a rate ranging from about 0.0001 lbs/day to about 0.1 lbs/day, from about 0.0002 lbs/day to about 0.09 lbs/day, from about 0.0003 lbs/day to about 0.1 lbs/day, from about 0.0004 lbs/day to about 0.09 lbs/day, from about 0.0005 lbs/day to about 0.08 lbs/day, from about 0.0006 lbs/day to about 0.07 lbs/day, from about 0.0007 lbs/day to about 0.6 lbs/day, from about 0.008 lbs/day to about 0.05 lbs/day, from about 0.009 lbs/day to about 0.04 lbs/day, from about 0.01 lbs/day to about 0.03 lbs/day, or about 0.001 lbs/day.

### Definitions

When introducing elements of the present disclosure, the articles “a,” “an,” “the,” and “said” are intended to mean that there are one or more of the elements. The use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms as used herein and in the claims shall include pluralities, and plural terms shall include the singular.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges can independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

As used herein, the terms “about” and “approximately” designate that a value is within a statistically meaningful range. Such a range can be typically within 20%, more typically still within 10%, and even more typically within 5% of a given value or range. The allowable variation encompassed by the terms “about” and “approximately” depends on the particular system under study and can be readily appreciated by one of ordinary skill in the art.

As used herein, “administering” is used in its broadest sense to mean contacting a subject with a composition disclosed herein, provided the method is a controlled method of administration capable of administering an accurate amount of therapeutic clay to the animal.

The phrases “effective amount” or “therapeutically effective amount” is used to mean an amount that is intended to qualify the amount of an agent or compound, that when administered, it will achieve the goal of improving the performance of an animal compared to the performance of the animal fed a control diet without the agent or compound.

As used herein, the term “w/w” designates the phrase “by weight” and is used to describe the concentration of a particular substance in a mixture or solution.

As used herein, the term “subject” refers to a vertebrate species such as mammals, birds, reptiles, amphibians, and fish. The vertebrate species may be an embryo, a juvenile, or an adult. Examples of suitable mammals include, without limit, rodents, companion or domestic animals, livestock, and primates. Non-limiting examples of rodents include mice, rats, hamsters, gerbils, and guinea pigs. Non-limiting examples of livestock include goats, sheep, swine, cattle, llamas, and alpacas. Suitable primates include, but are not limited to, humans, capuchin monkeys, chimpanzees, lemurs, macaques, marmosets, tamarins, spider monkeys, squirrel monkeys, and vervet monkeys. Non-limiting examples of birds include chickens, turkeys, ducks, and geese.

As used herein, the terms “companion animal” or “domestic animal” refer to an animal typically kept as a pet for keeping in the vicinity of a home or domestic environment for company or protection, regardless of whether the animal is kept indoors or outdoors. Non-limiting examples of companion animals or domestic animals include, but are not limited to, dogs, cats, house rabbits, ferrets, and horses.

As used herein, the term “adult” when referring to an animal refers to an animal at stage after the animal is weened and removed from the nursery. When the animal is a pig, the term “adult” refers to pigs about 8 weeks old and older.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. “Purify” or “purification” in other aspects means removing at least one contaminant from the composition to be purified. In this sense, purification does not require that the purified compound be homogenous, e.g., 100% pure.

As various changes could be made in the above-described cells and methods without departing from the scope of the disclosure, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Feeding Therapeutic Clay with and without Tribasic Copper Chloride (TBCC) to Grow-Finish Pigs on Growth Performance and Carcass Characteristics

A study was conducted to evaluate the effects of feeding the therapeutic clay of the instant disclosure with and without TBCC to healthy grow-finish pigs on growth performance and carcass characteristics. At arrival (˜35 lb), pigs were sorted into 27 pigs/pen and balanced as closely as possible on gender. Pens were fed a common diet until the start of the trial, approximately 2 to 3 weeks later. On the first day of the experiment, pens were weighed and blocked by average bodyweight, and pens within block were randomly assigned to one of 4 dietary treatments (1 control and 3 experimental diets; Table 1) in a randomized complete block design. This resulted in 12 pens for Trt 3 and 4 and 11 pens for Trt 1 and 2 for the evaluation of growth performance, carcass characteristics, and health status.

TABLE 1
Dietary treatment layout

| | | Inclusion | | | |
|---|---|---|---|---|---|
| | | Rate | # of | # | # of |
| Treatment # | Additive | Product | Pens | Pigs/pen | Animals |
| 1-Control | N/A | N/A | 11 | 27 | 297 |
| 2- | Therapeutic | 0.4 lb/ton | 11 | 27 | 297 |
| Therapeutic | clay | | | | |
| clay | | | | | |
| 3-TBCC | TBCC | 0.4 lb/ton | 12 | 27 | 324 |
| 4- | Therapeutic | 0.4 and 0.4 | 12 | 27 | 324 |
| Therapeutic | clay + TBCC | lb/ton, | | | |
| clay + BCC | | resp. | | | |
| | | | | Total | 1,238 |

On the first day of the experiment, each pen was identified by a unique EDC tag. Once a pen was scanned, pen weight, head count, feeder measurement, and any removals were recorded prior to scanning a new pen tag.

Pigs were weighed by pen every 2 weeks (+/−1 day) except between barn top and run out. Feed leftover was measured at the time each pen was weighed. This allowed for the calculation of ADG, ADFI, and F/G by pen. A minimum of 0.5 lb fresh fecal samples were collected from a minimum of 3 pigs per pen for every pen.

The composition of the basal diet is shown in Table 2 and each diet contained a unique treatment color micro-tracer at 10 g/ton for feed delivery monitoring. Feed was provided through the Feed Logic® system allowing collection of feed intake data by pen.

Data were analyzed using ANOVA by the MIXED procedure of SAS. For growth performance of grow-finish phase and carcass characteristics, pen served as the experimental unit. The statistical model included fixed effect of dietary treatments and random effect of block. Initial pen BW was used as covariate for analysis of growth performance. Multiple comparisons between treatments were performed using the Tukey adjustment option of SAS. All results were reported as least squares means. The significance level chosen was α=0.05. Treatment effect was considered significant if P<0.05, whereas values between 0.05≤P≤0.10 were considered as statistical trends.

Feeding Therapeutic clay during the first 8 weeks in the finishing period (63-195 lbs) resulted in reduced ADFI and reduced ADG (Table 3). Feed conversion was similar between Therapeutic clay and the control-fed pigs, indicating that the reduction in growth was a direct result of reduced feed intake. However, from weeks 9 to 17 (196 to 290 lbs) ADG was increased (P<0.05) by feeding Therapeutic clay even though feed intake was similar between control and Therapeutic clay fed pigs. Efficiency of nutrient utilization was improved and can partially be explained by the improvement in volatile fatty acid (VFA) production illustrated in Table 4. Pigs fed Therapeutic clay had increased (P<0.10) total fecal VFA concentrations compared to control-fed pigs. An interaction effect was seen with tribasic copper chloride, where Therapeutic clay numerically increased VFA production in control diets without tribasic copper chloride addition but reduced the VFA concentrations in pigs fed diets with tribasic copper chloride. The 12% increase in VFA production for pigs fed Therapeutic clay, results in greater energy release from the diet that can be used for intestinal maintenance. This energy generation in the large intestine can serve as maintenance energy for the digestive tract, increasing the efficiency of dietary energy for growth.

TABLE 2
Composition of the basal diet (Treatment 1-Control)

| | 2. Basal Diets | | | | | | | |
|---|---|---|---|---|---|---|---|---|
| | | | 5. 95-135 | | | 9. 190-215 | | |
| 1. Ingredient, | 3. 55-75 | 4. 75-95 | NQ3 | 7. 135-165 | 8. 165-190 | NQ6 | 11. 215-250 | 12. 250-290 |
| lb/ton | NQ1 | NQ2 | 6. Den/CTC | NQ4 | NQ5 | 10. Den | NQ7 | NQ8 |
| 13. corn | 14. 940.06 | 15. 1004.52 | 16. 1061.72 | 17. 1124.88 | 18. 1170.33 | 19. 1202.08 | 20. 1466.95 | 21. 1494.99 |
| 22. Soybean | 23. 323.00 | 24. 259.00 | 25. 193.00 | 26. 137.00 | 27. 97.00 | 28. 64.00 | 29. 121.00 | 30. 95.00 |
| 46% | | | | | | | | |
| 31. Lysine 98.5 | 32. 12.40 | 33. 12.10 | 34. 11.60 | 35. 11.10 | 36. 10.80 | 37. 10.60 | 38. 8.70 | 40. 8.50 |
| 40. Methionine | 41. 2.00 | 42. 1.50 | 43. 0.95 | 44. 0.45 | 45. 0.20 | 46. N/A | 47. N/A | 48. N/A |
| 99% DL | | | | | | | | |
| 49. Threonine | 50. 2.95 | 51. 2.65 | 52. 2.50 | 53. 2.15 | 54. 1.95 | 55. 1.90 | 56. 2.15 | 57. 2.10 |
| 98.5% | | | | | | | | |
| 58. Tryptophan | 59. 0.92 | 60. 0.96 | 61. 0.96 | 62. 0.95 | 63. 0.95 | 64. 0.95 | 65. 0.73 | 66. 0.74 |
| 98.5% | | | | | | | | |
| 67. Limestone | 68. 24.30 | 69. 23.90 | 70. 24.10 | 71. 25.20 | 72. 2.50 | 73. 20.80 | 74. 19.30 | 75. 18.50 |
| 76. Salt | 77. 10.00 | 78. 10.00 | 79. 10.00 | 80. 10.00 | 81. 10.00 | 82. 10.00 | 83. 10.00 | 84. 10.00 |
| 85. Optiphos 2000 | 86. 0.35 | 87. 0.35 | 88. 0.35 | 89. 0.35 | 90. 0.35 | 91. 0.35 | 92. 0.35 | 93. 0.35 |
| 94. VTM | 95. 2.00 | 96. 2.00 | 97. 1.90 | 98. 1.90 | 99. 1.90 | 100. 1.80 | 101. 1.80 | 102. 1.80 |
| 103. Distillers | 104. 600.00 | 105. 600.00 | 106. 600.00 | 107. 600.00 | 108. 600.00 | 109. 600.00 | 110. 300.00 | 111. 300.00 |
| Dried grains | | | | | | | | |
| 112. Tallow | 113. 82.00 | 114. 83.00 | 115. 85.00 | 116. 86.00 | 117. 84.00 | 118. 84.00 | 119. 68.00 | 120. 67.00 |
| 121. Lipinate | 122. N/A | 123. N/A | 124. N/A | 125. N/A | 126. N/A | 127. N/A | 128. 1.00 | 129. 1.00 |
| 130. CTC- | | | | | | | | |
| Aureomycin 90 | 131. N/A | 132. N/A | 133. 4.40 | 134. N/A | 135. N/A | 136. N/A | 137. N/A | 138. N/A |
| 139. Denagard 10 | 140. N/A | 141. N/A | 142. 3.50 | 143. N/A | 144. N/A | 145. 3.50 | 146. N/A | 147. N/A |
| 148. Microtracer | 149. 0.02 | 150. 0.02 | 151. 0.02 | 152. 0.02 | 153. 0.02 | 154. 0.02 | 155. 0.02 | 156. 0.02 |
| 157. Total | 158. 2000.00 | 159. 2000.00 | 160. 2000.00 | 161. 2000.00 | 162. 2000.00 | 163. 2000.00 | 164. 2000.00 | 165. 2000.00 |
| 166. Feed Budget | 167. 42 | 168. 37 | 169. 90 | 170. 69 | 171. 69 | 172. 72 | 173. 109 | 174. 145 |
| 175. Days on Feed | 176. 12 | 177. 9 | 178. 18 | 179. 12 | 180. 11 | 181. 11 | 182. 16 | 183. 21 |

184. Nutrient Composition

| | | | | | | | | |
|---|---|---|---|---|---|---|---|---|
| 185. Mod ME, | 186. 1519.00 | 187. 1521.00 | 188. 1526.00 | 189. 1529.00 | 190. 1531.00 | 191. 1533.00 | 192. 1540.00 | 193. 1541.00 |
| kcal/lb | | | | | | | | |
| 194. SID Lysine, | 195. 3.47 | 196. 3.19 | 197. 2.89 | 198. 2.61 | 199. 2.43 | 200. 2.28 | 201. 2.12 | 202. 2.00 |
| g/mcal | | | | | | | | |
| 203. SID Thr/Ly, % | 204. 0.62 | 205. 0.62 | 206. 0.63 | 207. 0.63 | 208. 0.63 | 209. 0.64 | 210. 0.65 | 211. 0.66 |
| 212. SID M + C/Lys, | 213. 0.57 | 214. 0.57 | 215. 0.57 | 216. 0.57 | 217. 0.57 | 218. 0.58 | 219. 0.57 | 220. 0.58 |
| % | | | | | | | | |
| 221. SID Try/Lys, | 222. 0.18 | 223. 0.18 | 224. 0.18 | 225. 0.18 | 226. 0.18 | 227. 0.18 | 228. 0.18 | 229. 0.18 |
| % | | | | | | | | |
| 230 SID Iso/Lys, % | 231. 0.55 | 232. 0.55 | 233. 0.55 | 234. 0.55 | 235. 0.55 | 236. 0.55 | 237. 0.55 | 238. 0.55 |
| 239. SID Val/Lys, | 240. 0.65 | 241. 0.65 | 242. 0.65 | 243. 0.70 | 244. 0.71 | 245. 0.72 | 246. 0.69 | 247. 0.70 |
| % | | | | | | | | |
| 248. Protein, % | 249. 20.69 | 250. 19.38 | 251. 18.04 | 252. 16.85 | 253. 16.05 | 254. 15.39 | 255. 13.62 | 256. 13.11 |
| 257. SID Lysine, % | 258. 1.16 | 259. 1.07 | 260. 0.97 | 261. 0.88 | 262. 0.82 | 263. 0.77 | 264. 0.72 | 265. 0.68 |
| 266. Avail Phos | 267. 0.37 | 268. 0.37 | 269. 0.36 | 270. 0.36 | 271. 0.36 | 272. 0.35 | 273. 0.27 | 274. 0.26 |
| Equil, % | | | | | | | | |
| 275. Calcium, % | 276. 0.70 | 277. 0.68 | 278. 0.67 | 279. 0.68 | 280. 0.62 | 281. 0.58 | 282. 0.56 | 283. 0.54 |
| 284. Anzl Calcium, | 285. 0.56 | 286. 0.54 | 287. 0.53 | 288. 0.54 | 289. 0.48 | 290. 0.44 | 291. 0.42 | 292. 0.40 |
| % | | | | | | | | |
| 293. Phosphorus, | 294. 0.50 | 295. 0.48 | 296. 0.47 | 297. 0.46 | 298. 0.45 | 299. 0.44 | 300. 0.36 | 301. 0.36 |
| % | | | | | | | | |
| 302. aP Release, % | 303. 0.13 | 304. 0.13 | 305. 0.13 | 306. 0.13 | 307. 0.13 | 308. 0.13 | 309. 0.13 | 310. 0.13 |
| 311. AOAC | 312. 875.00 | 313. 875.00 | 314. 875.00 | 315. 875.00 | 316. 875.00 | 317. 875.00 | 318. 875.00 | 319. 875.00 |
| PHYTASE, | | | | | | | | |
| FTU/kg | | | | | | | | |
| 320. Salt, % | 321. 0.50 | 322. 0.50 | 323. 0.50 | 324. 0.50 | 325. 0.50 | 326. 0.50 | 327. 0.50 | 328. 0.50 |
| 329. Fat, % | 330. 8.27 | 331. 8.92 | 332. 8.88 | 333. 9.03 | 334. 8.92 | 335. 7.68 | 336. 7.35 | 337. 7.35 |
| (approximate) | | | | | | | | |

TABLE 3
Effect of feeding Therapeutic clay with and without tribasic copper chloride (TBCC) on growth
performance in pigs from 60 to 290 lb

| | Main effect | | Main effect | | | | | | | | | |
|---|---|---|---|---|---|---|---|---|---|---|---|---|
| | of TBCC | | of Clay | | No TBCC | | TBCC | | | P-value | | |
| | No | | No | | No | | No | | | | | Inter- |
| Item | TBCC | TBCC | Clay | Clay | Clay | Clay | Clay | Clay | PSE | TBCC | Clay | action |
| # of Pens | 22 | 24 | 23 | 23 | 11 | 11 | 12 | 12 | | | | |
| # of Pigs | 592 | 646 | 619 | 619 | 296 | 296 | 323 | 323 | | | | |
| Start BW, lb | 62.7 | 62.8 | 62.7 | 62.7 | 62.7 | 62.7 | 62.8 | 62.8 | 1.1 | 0.92 | 0.99 | 0.98 |
| BW end of | 163.9 | 166.4 | 166.7 | 163.6 | 165.9 | 161.9 | 167.5 | 165.3 | 1.1 | 0.03 | 0.007 | 0.43 |
| Week 8, lb | | | | | | | | | | | | |
| BW end of | 288.0 | 290.1 | 288.4 | 289.7 | 287.6 | 288.4 | 289.2 | 291.0 | 2.1 | 0.31 | 0.53 | 0.82 |
| Week 17, lb | | | | | | | | | | | | |
| Week 1-8 | | | | | | | | | | | | |
| ADG, lb/day | 1.80 | 1.85 | 1.85 | 1.80 | 1.84 | 1.76 | 1.87 | 1.83 | 0.02 | 0.02 | 0.006 | 0.34 |
| ADFI, lb/day | 3.82 | 3.92 | 3.93 | 3.82 | 3.92^{a} | 3.72^{b} | 3.93^{a} | 3.91^{a} | 0.04 | 0.02 | 0.01 | 0.06 |
| F:G | 2.12 | 2.12 | 2.12 | 2.12 | 2.13 | 2.11 | 2.11 | 2.14 | 0.02 | 1.00 | 0.86 | 0.20 |
| Week 9-17 | | | | | | | | | | | | |
| ADG, lb/day | 1.98 | 1.97 | 1.94 | 2.01 | 1.94 | 2.02 | 1.95 | 2.00 | 0.02 | 0.74 | 0.002 | 0.38 |
| ADFI, lb/day | 5.95 | 5.95 | 5.93 | 5.97 | 5.97 | 5.93 | 5.89 | 6.01 | 0.06 | 0.99 | 0.54 | 0.22 |
| F:G | 3.01 | 3.02 | 3.06 | 2.97 | 3.09^{a} | 2.93^{b} | 3.03^{a} | 3.01^{ab} | 0.03 | 0.88 | 0.001 | 0.009 |
| Overall; | | | | | | | | | | | | |
| Week 1-17 | | | | | | | | | | | | |
| ADG, lb/day | 1.89 | 1.91 | 1.90 | 1.91 | 1.89 | 1.89 | 1.91 | 1.92 | 0.01 | 0.16 | 0.60 | 0.92 |
| ADFI, lb/day | 4.91 | 4.97 | 4.96 | 4.92 | 4.98^{xy} | 4.84^{x} | 4.94^{xy} | 5.00^{y} | 0.05 | 0.20 | 0.42 | 0.03 |
| F:G | 2.60 | 2.60 | 2.61 | 2.58 | 2.64^{a} | 2.56^{b} | 2.59^{ab} | 2.61^{ab} | 0.02 | 0.97 | 0.05 | 0.002 |
| Total removal, | 9.0 | 6.8 | 7.9 | 7.8 | 6.1^{ab} | 11.8^{c} | 9.6^{bc} | 4.0^{a} | N/A | 0.18 | 0.92 | N/A |
| % | | | | | | | | | | | | |
| Mortality, % | 2.7 | 2.6 | 3.2 | 2.1 | 2.0^{ab} | 3.4^{a} | 4.3^{a} | 0.9^{b} | N/A | 0.94 | 0.22 | N/A |
| Fallbacks, % | 3.4 | 1.9 | 2.3 | 2.9 | 2.0^{ab} | 4.7^{b} | 2.5^{ab} | 1.2^{a} | N/A | 0.10 | 0.48 | N/A |
| Twisted gut, % | 0.8 | 1.9 | 2.3 | 0.5 | 0.7^{ab} | 1.0^{b} | 3.7^{c} | 0.0^{a} | N/A | 0.13 | 0.008 | N/A |
| Ulcer, % | 2.0 | 1.1 | 1.3 | 1.8 | 1.0^{ab} | 3.0^{b} | 1.5^{ab} | 0.6^{a} | N/A | 0.18 | 0.49 | N/A |
| Carcass | | | | | | | | | | | | |
| characteristics | | | | | | | | | | | | |
| HCW, lb | 221.0 | 220.8 | 220.4 | 221.4 | 220.9 | 221.1 | 219.8 | 221.7 | 1.8 | 0.91 | 0.58 | 0.65 |
| Carcass yield, | 76.7 | 76.1 | 76.4 | 76.4 | 76.8 | 76.6 | 76.0 | 76.2 | 0.3 | 0.07 | 0.98 | 0.55 |
| % | | | | | | | | | | | | |
| Backfat | 17.1 | 17.8 | 17.5 | 17.3 | 17.1 | 17.1 | 17.9 | 17.6 | 0.3 | 0.02 | 0.48 | 0.56 |
| thickness, mm | | | | | | | | | | | | |
| Loin depth, mm | 59.2 | 62.6 | 61.4 | 60.4 | 59.4 | 59.0 | 63.5 | 61.7 | 1.8 | 0.06 | 0.57 | 0.69 |
| Carcass lean, % | 55.5 | 55.5 | 55.5 | 55.5 | 55.6 | 55.5 | 55.5 | 55.6 | 0.2 | 1.00 | 0.99 | 0.91 |
| Carcass ADG, | 1.44 | 1.44 | 1.44 | 1.45 | 1.44 | 1.44 | 1.43 | 1.45 | 0.02 | 0.91 | 0.58 | 0.65 |
| lb/day | | | | | | | | | | | | |
| Carcass F/G | 3.35 | 3.39 | 3.39 | 3.35 | 3.40 | 3.30 | 3.38 | 3.40 | 0.03 | 0.26 | 0.20 | 0.07 |

^{a,b,c}Means without a common superscript differ (P < 0.05)
^{x,y}Means without a common superscript tend to differ (P < 0.10)

TABLE 4
Feeding Therapeutic clay with and without TBCC on volatile fatty acids production in feces

| | Main effect | | Main effect | | | | | | | | | |
|---|---|---|---|---|---|---|---|---|---|---|---|---|
| | of TBCC | | of Clay | | No TBCC | | TBCC | | | | | |
| | No | | No | | No | | No | | | P-value | | |
| Item | TBCC | TBCC | Clay | Clay | Clay | Clay | Clay | Clay | PSE | TBCC | Clay | Interaction |

VFA concentrations in feces, μmol/g

| | | | | | | | | | | | | |
|---|---|---|---|---|---|---|---|---|---|---|---|---|
| Acetate | 126.0 | 143.5 | 134.1 | 135.4 | 120.8^{a} | 131.2^{ab} | 147.3^{b} | 139.7^{b} | 5.2 | 0.002 | 0.79 | 0.09 |
| Propionate | 78.8 | 86.1 | 81.8 | 83.2 | 75.6 | 82.1 | 88.1 | 84.2 | 4.5 | 0.11 | 0.76 | 0.25 |
| Butyrate | 58.1 | 67.8 | 59.8 | 66.0 | 50.5^{a,x} | 65.6^{y} | 69.2^{b} | 66.4^{y} | 4.5 | 0.04 | 0.18 | 0.05 |
| Isovalerate | 8.7 | 9.7 | 8.7 | 9.6 | 7.6 | 9.8 | 9.9 | 9.4 | 0.8 | 0.25 | 0.31 | 0.11 |
| Total VFA | 277.0 | 313.0 | 289.9 | 300.1 | 259.3^{a} | 294.7^{ab} | 320.4^{b} | 305.5^{ab} | 14.2 | 0.02 | 0.48 | 0.09 |

^{a,b,c}Means without a common superscript differ (P < 0.05)
^{x,y}Means without a common superscript tend to differ (P < 0.10)

### Example 2. Meta-Analysis of Four Therapeutic Clay Grow-Finish Studies

Four experiments were conducted following the same procedure shown in Example 1 to evaluate the effect of feeding Therapeutic clay on growth performance of finishing pigs. In each experiment, finishing pigs (start BW=200 lb) were housed in pens (27 pigs per pen) and randomly assigned to either a control diet (CON) or a diet containing Therapeutic clay at 0.4 lb/ton, resulting in 44 replicated pens for CON and 47 replicated pens for Therapeutic clay treatment over three experiments. Pigs were fed their respective experimental diets for an average of 44 d prior to harvest. Study 1, 3 and 4 did not have TBCC in the basal diet, while Study 2 had TBCC at 0.8 lb/ton to provide 150 ppm of Cu of the diet. Data from the three experiments were compiled for meta-analysis using the MIXED procedure of SAS.

Data in Table 5 suggest that feeding pigs a diet containing Therapeutic clay from 200 to 290 lbs had improved (P<0.05) ADG and feed conversion compared to pigs fed the control diet. Gain was improved as a result of improved feed conversion as feed intake was similar for pigs fed diets containing Therapeutic clay and pigs fed the control diet. Ending body weight tended to increase (P=0.07) in pigs fed diets containing Therapeutic clay compared to pigs fed control diets. Caloric efficiency was improved (P<0.05) for pigs fed diets including therapeutic clay compared to control fed pigs.

TABLE 5
Meta-analyses of effect of feeding Therapeutic clay on growth
performance in pigs from 200 to 290 lbs

| | Meta-analysis | | | | |
|---|---|---|---|---|---|
| | | Therapeutic | | | % |
| Item | Control | clay | SE | P-value | Change |
| # of Trials | 4 | 4 | | | |
| # of Pens | 44 | 47 | | | |
| # of Pigs | 1187 | 1268 | | | |
| Days on | 44 | 44 | | | |
| experiment | | | | | |
| Start BW, lb | 201.0 | 199.3 | 0.6 | 0.05 | |
| ADG, lb/day | 1.99 | 2.04 | 0.01 | 0.01 | 2.2% |
| ADFI, lb/day | 6.22 | 6.22 | 0.04 | 0.96 | 0.0% |
| F/G | 3.13 | 3.06 | 0.01 | 0.002 | −2.1% |
| End BW, lb | 285.0 | 286.4 | 0.6 | 0.07 | 0.5% |
| Caloric | 4818 | 4717 | 22 | 0.001 | −2.1% |
| efficiency, | | | | | |
| kcal/lb gain | | | | | |

### Example 3. Using Energy Prediction Equation to Predict Feed Intake and Feed Conversion in Finishing Pigs Fed Therapeutic Clay

Linear regression analysis was conducted to evaluate the energy partitioning between maintenance and gain of pigs fed the Control diet and the diet containing Therapeutic clay using the data generated from four experiments mentioned in Example 2. The following linear equation was fitted in each study to predict feed intake to reach the requirement for maintenance and gain in pigs fed Control and Therapeutic clay diets separately:

ADFI=β₀+β₁BW^{\hat{ }}0.66+β₂ADG+β₃Trt+β₄(BW^{\hat{ }}0.66×Trt)+β₅(ADG×Trt)

in which the term BW^{\hat{ }}0.66=main effect of energy requirement for maintenance, ADG=main effect of energy requirement for gain, Trt=main effect of dietary treatment, BW^{\hat{ }}0.66×Trt=interaction between dietary treatments and energy requirement for maintenance, and ADG×Trt=interaction between dietary treatments and energy for gain. The General Linear Models procedure (PROC GLM) in SAS software was used to solve each coefficient of this model for each study. The interaction terms (BW^{\hat{ }}0.66×Trt) and (ADG×Trt) were dropped in Study 1, 2 and 4 since they were not significant (P>0.10). The final models used in Study 1, 2 and 4 were:

ADFI=β₀+β₁BW^{\hat{ }}0.66+β₂ADG+β₃Trt

The prediction equations of ADFI in each study are shown below:

ADFI(Control)=−3.133+0.1745×BW^{\hat{ }}0.66+1.386×ADG+0.158

ADFI(Therapeutic clay)=−3.133+0.1745×BW^{\hat{ }}0.66+1.386×ADG, with R²=0.56 Study 1:

ADFI(Control)=−2.774+0.147×BW^{\hat{ }}0.66+1.589×ADG−0.102

ADFI(Therapeutic clay)=−2.774+0.147×BW^{\hat{ }}0.66+1.589×ADG, with R²=0.76 Study 2:

ADFI(Control)=−4.517+0.252×BW^{\hat{ }}0.66+0.734×ADG+1.371−0.126×BW^{\hat{ }}0.66+1.752×ADG

ADFI(Therapeutic clay)=−4.517+0.252×BW^{\hat{ }}0.66+0.734×ADG, with R²=0.78 Study 3:

ADFI(Control)=−1.648+0.141×BW^{\hat{ }}0.66+1.229×ADG+0.147

ADFI(Therapeutic clay)=−1.648+0.141×BW^{\hat{ }}0.66+1.229×ADG, with R²=0.56 Study 4:

The differences in predicted average daily feed intake and feed to gain ratio between Control and Therapeutic clay treatments were calculated using the above prediction equations with the assumption of 2.20 lb/day of ADG for pigs at 250 lb of BW (Table 6).

TABLE 6
Differences in predicted average daily feed intake and feed
to gain ratio between Control and Therapeutic clay treatments
assuming ADG of 2.20 lb/day for pigs at 250 lb of BW

| | Difference in predicted | Difference in |
|---|---|---|
| Study | ADFI, lb/day | predicted F/G |
| Study 1 | −0.16 | −0.07 |
| Study 2 | +0.10 | +0.05 |
| Study 3 | −0.39 | −0.18 |
| Study 4 | −0.15 | −0.07 |

In addition, Study 3 also showed significant interactions of (BW^{\hat{ }}0.660.66×Trt) and (ADG×Trt), suggesting that the effects of feeding Therapeutic clay on dietary energy requirement for maintenance and gain may be dependent on pig BW and growth rate. To further determine the correlation between pig BW and growth rate on reduction of dietary energy requirements in pigs fed Therapeutic clay, the difference between the predicted ADFI for pigs fed Control and Therapeutic clay was calculated as:

Difference in ADFI(Therapeutic clay−Control)=−1.371+0.126×BW^{\hat{ }}0.66−1.752×ADG

It suggests that the reduction in dietary energy requirement, as indicated by difference in ADFI (Therapeutic clay−Control), would be greater in lighter pigs and pigs with faster growth rate. The lack of response in pigs less than 180 lbs illustrates that change in energy availability becomes relevant as the pig matures and organ weights increase. These data suggest that improved intestinal performance result in less energy for maintenance and improving pig gain. This effect was not seen in younger, lighter pigs which use energy more efficiently.

### Example 4. Mode of Action of Improved Intestinal Health and Feed Efficiency in Grow-Finish Pigs Fed Therapeutic Clay

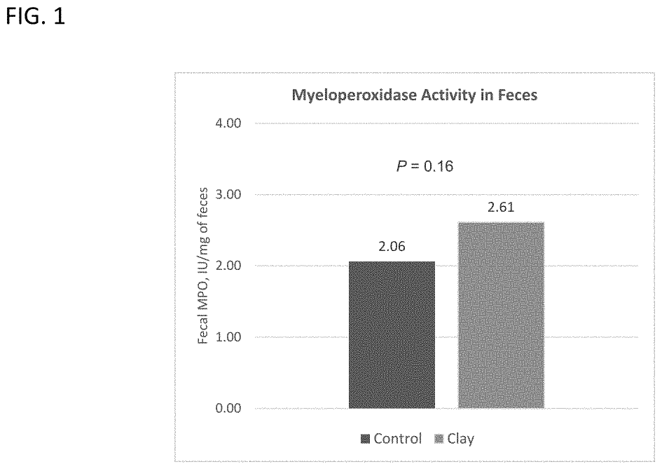

The mode of action for reduced maintenance energy requirements and feed efficiency were further evaluated. Fecal myeloperoxidase (MPO) is the major protein in neutrophil cells and has been used as biomarker to indicate intestinal inflammation in humans and pigs. Pigs fed Therapeutic clay had similar enzyme activity of fecal MPO as those fed Control diet (FIG. 1), suggesting that the mode of action of Therapeutic clay that improves intestinal health and feed utilization is not likely due to the anti-inflammation or antimicrobial effect in the gastrointestinal tract.

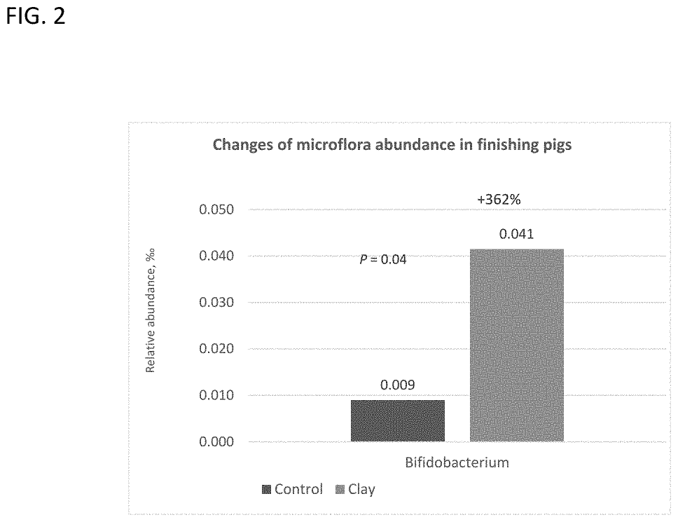

A full-length 16S ribosomal RNA (rRNA) sequencing was conducted in the fecal microbiome of finishing pigs fed Control and therapeutic therapeutic clay diets to evaluate the microbial profiling. Pigs fed a Therapeutic therapeutic clay diet compared to pigs fed a diet containing control diet showed significantly higher relative abundance (P=0.04) of Bifidobacterium, which is the leading probiotic species in grow-finish pigs (FIG. 2). In our previous study where Therapeutic therapeutic clay was fed to younger and lighter pigs, the abundance of Bifidobacterium in feces was not changed.

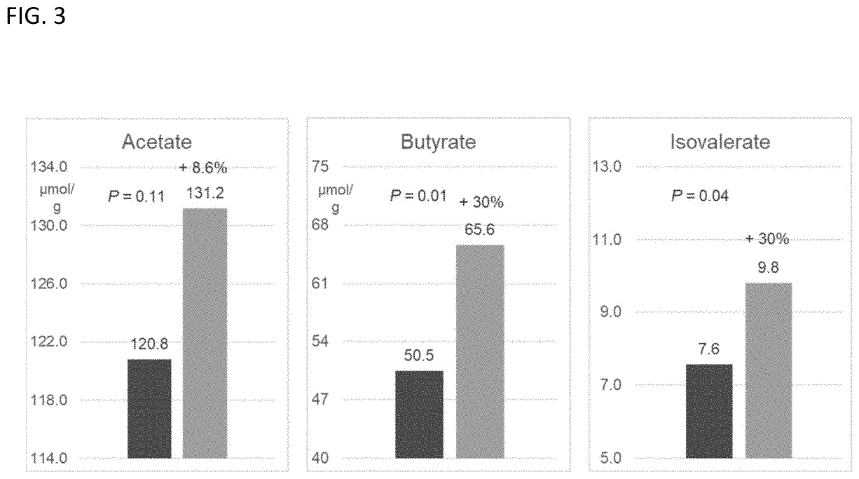

The changes on microflora composition from feeding Therapeutic therapeutic clay and the shift to more beneficial bacteria species may also lead to several other positive biological impact. For example, finishing pigs fed diet containing Therapeutic therapeutic clay also showed 8.6% increase of acetate acid (P=0.11), 30% increase of butyrate acid (P=0.01) and 30% increase of isovalerate acid (P=0.04) in feces compared to pigs fed Control diet (FIG. 3). The increased production of these short-chain fatty acids results in greater energy release that can be used for maintenance and growth, which reduce dietary energy requirements, improves energy for gain and ultimately improves feed efficiency.

Taken together the data in the Examples above illustrate that pigs fed diets containing Therapeutic clay, from 165 to 290 lbs BW, had reduced maintenance energy requirements, resulting in greater energy for body weight gain and improved feed conversion. Feeding diets containing bentonite therapeutic clay did not result in the same type of improvements as feeding diets containing Therapeutic clay to pigs from 165 to 290 lbs. The improved intestinal health results in less dietary energy required for maintenance, improved feed conversion, or both in the grow-finish pigs. The reduced dietary energy requirement for maintenance and can be attributed to increased abundance of beneficial bacteria that produce increased fecal volatile fatty acids concentrations that can be used as energy for maintenance in the intestine, decreasing the amount of energy needed from the diet. Further, the ability to ferment fiber in a finishing pig is greater than in a nursery pig. Therefore, the reduced dietary energy requirement for maintenance and gain in grow-finish pigs when compared to nursery pigs can also be attributed to the increase in the ability to ferment fiber with age and maturity in nonruminants.

What is claimed is:

1. A method of improving performance of an adult animal, the method comprising orally administrating to the animal a therapeutically effective amount of a therapeutic clay.

2. The method of claim 1, wherein the therapeutic clay is clay mined in the Crater Lake region of the Cascade Mountains of Oregon.

3. The method of claim 1, wherein the therapeutic clay is naturally mined, and the level of reducing agent in the clay is adjusted to provide therapeutically effective amounts of the reducing agent.

4. The method of claim 1, wherein the adult animal is an adult livestock animal.

5. The method of claim 1, wherein the adult animal is a grow-finish pig.

6. The method of claim 1, wherein the adult animal is a healthy grow-finish pig.

7. The method of claim 6, wherein the therapeutic clay is administered to the pig at or after about week 6 or at about week 8 to about week 17 of growing and finishing.

8. The method of claim 6, wherein the clay is fed to the pig when the weight of the pig is about 160 to about 200 lbs or about 190 to about 200 lbs.

9. The method of claim 1, wherein improving performance comprises reducing maintenance nutrient requirements, improving growth performance, reducing the energy required for intestinal maintenance, reducing the energy required for weight gain, reduced morbidity, increasing growth rate, improving feed conversion, improving the average daily gain (ADG), improving the efficiency of nutrient utilization, increasing volatile fatty acid production, improving energy use from volatile fatty acids, increasing ending body weight, improved intestinal health, increased relative abundance of Bifidobacterium in the intestines, or combinations thereof.

10. The method of claim 1, wherein the clay is formulated in a feed composition for oral administration to the animal.

11. The method of claim 10, wherein the amount of clay in a feed composition ranges from about 0.005% to about 0.1%, from about 0.01% to about 0.05%, or about 0.01% to about 0.02% of the feed composition.

12. The method of claim 10, wherein the amount of clay in a feed composition ranges from about 0.01 to about 10 lbs/ton, from about 0.1 to about 1 lbs/ton, or about 0.2 to about 0.6 lbs/ton of the feed composition.

13. A feed composition for improving performance of an adult animal, the composition comprising a therapeutically effective amount of a therapeutic clay.

14. The feed composition of claim 13, wherein the therapeutic clay is clay mined in the Crater Lake region of the Cascade Mountains of Oregon.

15. The feed composition of claim 13, wherein the therapeutic clay is naturally mined, and the level of reducing agent in the clay is adjusted to provide antimicrobial effective amounts of the reducing agent.

16. The feed composition of claim 13, wherein the adult animal is an adult livestock animal.

17. The feed composition of claim 13, wherein the adult animal is a healthy grow-finish pig.

18. The feed composition of claim 17, wherein feed composition is a basal feed composition.

19. The feed composition of claim 17, wherein the amount of clay in a feed composition ranges from about 0.005% to about 0.1%, from about 0.01% to about 0.05%, or about 0.01% to about 0.02% of the feed composition.

20. The feed composition of claim 17, wherein the amount of clay in a feed composition ranges from about 0.01 to about 10 lbs/ton, from about 0.1 to about 1 lbs/ton, or about 0.2 to about 0.6 lbs/ton of the feed composition.
